(19) United States
(12) Patent Application Publication
(10) Pub. No.: US 2025/0368935 A1
(43) Pub. Date: Dec. 4, 2025

(54) HIGH-SPEED CELL CULTURE SUBSTRATE SUITABLE FOR STERILIZATION AND CELL SEPARATION PROCESSES

(71) Applicant: LMK CO., LTD,, Gumi-si (KR)

(72) Inventors: Sung il KIM, Pyeongtaek-si (KR); Hyun Sik MYUNG, Gumi-si (KR); Jong Geun LEE, Pohang-si (KR); Kyo Wung LEE, Pohang-si (KR)

(73) Assignee: LMK CO., LTD,, Gumi-si (KR)

(21) Appl. No.: 18/678,433

(22) Filed: May 30, 2024

(51) Int. Cl. C12M 1/12; C01B 32/205; C12M 1/00

(52) U.S. Cl. CPC C12M 25/14; C01B 32/205; C12M 29/04; C12M 37/00

(57) ABSTRACT

Proposed is a cell culture substrate suitable for sterilization and cell separation processes. The cell culture substrate includes a base, and a culture layer formed on the base and having a ‘cell contact surface’ which is a surface to which cells come into contact or attach. In the culture layer, a portion including at least the cell contact surface has a ‘first carbon allotrope portion’ which is an area made of a carbon allotrope. The first carbon allotrope portion is formed by physical vapor deposition (PVD). The first carbon allotrope portion has a higher hardness than graphite, which is another carbon allotrope, and a lower hardness than ‘PVD-DLC’ formed by PVD, which is a diamond-like carbon (DLC) and another carbon allotrope.

### BACKGROUND OF THE DISCLOSURE

### 1. Field of the Disclosure

The present disclosure relates to a substrate for cell culture and, more specifically, to a high-speed cell culture substrate capable of significantly enhancing cell culturability while preventing damage to a carbon culture layer during sterilization and cell separation processes.

### 2. Description of the Related Art

As cultured cells are increasingly used in diverse applications, such as in cell-cultured meat and disease treatment, interest in and research on cell culture is growing. Cell culture is a technique of collecting cells from living organisms and culturing the cells outside the living body. Cultured cells may be differentiated into various tissues of the body, such as skin, organs, and nerves, and then transplanted into the human body. Alternatively, cultured cells may be transplanted in a pre-differentiated state, thereby the cultured cells may engraft and differentiate simultaneously. Therefore, cultured cells have the potential to treat various diseases.

In particular, recently, research on cell-cultured meat has been actively conducted to create alternative protein meat by extracting and cultivating stem cells instead of raising animals. For the mass commercialization/commodification of such cell-cultured meat products, technology capable of maximizing the proliferation rate of cells is required.

Meanwhile, human or mammalian cells are known to exhibit two puzzling properties. The first is that human or mammalian cells do not replicate in in-vivo and tissue culture environments, and the second is that human or mammalian cells do not adhere well to conventional cell culture surfaces.

To solve these problems, there is a method of performing surface treatment on a cell culture substrate. Cell culture substrates for mammalian cell culture and analysis are typically containers or plates made of polymer or glass. To the surface of such containers or plates is surface treatment required to enable cells to adhere well.

Such surface treatment may be performed through chemical modification on the surface of the containers or plates. Chemical modification may include atmospheric corona, radio frequency vacuum plasma, DC glow discharge, and chemical-physical vapor deposition.

However, surface treatment methods for such cell culture substrates, such as surface modification, have limitations in significantly enhancing cell culturability, including cell proliferation and growth rates.

To overcome these limitations, techniques to enhance cell culturability have been proposed and applied to the cell culture substrate. One is a diamond-like carbon (DLC) application. The other one is the application of carbon-based materials, such as a two-dimensional carbon film with a graphite-like structure.

In other words, when cells are cultured with the conventionally proposed carbon-based cell culture substrates, applying a DLC carbon film on a substrate for coating or forming a two-dimensional (2D) carbon monolayer is performed, and then this prepared carbon film or carbon layer serves as a culture substrate.

Meanwhile, the cell culture process may involve a sterilization process to remove microorganisms such as bacteria and mold in the culture layer before culturing the cells and a cell separation process to separate the cells from the substrate.

However, when the culture layer of the cell culture substrate is formed as carbon-based materials, the high temperature/high pressure environment or chemicals used in the sterilization process may cause ‘culture layer damage’ such as cracks, delamination, and pinholes in the carbon culture layer.

In addition, when the carbon culture layer is exposed to trypsin during the cell separation process, trypsin may have negative effects such as damage, cracks, or peeling on the carbon culture layer. That is, during the cell separation process, trypsin decomposes proteins on the surface of the carbon culture layer, and as a result, damage to the structure of the carbon membrane, cracks, and detachment may occur.

Likewise, when the carbon culture layer is damaged during the sterilization and cell separation processes, carbon particles leaked from the carbon culture layer may enter and contaminate the cultured cells.

Therefore, when the carbon culture layer of the cell culture substrate is formed as carbon-based materials, it is important to enhance cell culturability, but it is also very important to prevent the carbon culture layer damage during the sterilization and cell separation processes.

### RELATED ART DOCUMENT

### Patent Document

(Patent document 0001) Korean Patent Application Publication No. 10-2007-0030780 (published on Mar. 16, 2007)

### SUMMARY OF THE DISCLOSURE

To solve the problems, the present disclosure is to greatly enhance cell culturability, including cell growth, proliferation rate, and cell adhesion, and to provide a high-speed cell culture substrate suitable for sterilization and cell separation processes to easily provide a substrate for cell culture.

The present disclosure also pertains to a high-speed cell culture substrate suitable for sterilization and cell separation processes. This cell culture substrate can enhance cell culturability compared to conventional substrates, and specifically the cell culture substrate is capable of preventing carbon culture layer damage, such as cracks, delamination, and pinholes, during sterilization and cell separation processes.

A high-speed cell culture substrate suitable for the sterilization and cell separation processes according to the present disclosure to achieve the purposes includes a base; and a culture layer formed on the base and having a ‘cell contact surface’ which is a surface to which cells come into contact or attach. In the culture layer, the area including at least the cell contact surface has a ‘first carbon allotrope portion’ which is a portion made of a carbon allotrope.

The first carbon allotrope portion is formed by physical vapor deposition (PVD). The first carbon allotrope portion has a higher hardness than graphite, which is another carbon allotrope, and a lower hardness than ‘PVD-DLC’ formed by PVD, which is a diamond-like carbon (DLC) and another carbon allotrope.

According to one embodiment of the present disclosure, the first carbon allotrope portion may have a hardness in a range of 4 to 29 Gpa.

According to another embodiment, the first carbon allotrope portion may have a carbon single bond ratio higher than the graphite and lower than the PVD-DLC.

The first carbon allotrope portion may have a carbon double bond ratio lower than the graphite and higher than the PVD-DLC.

According to yet another embodiment, the first carbon allotrope portion has a plurality of pores. The first carbon allotrope portion may be a material with a larger total pore volume than the graphite (or the PVD-DLC) when the first carbon allotrope portion and the graphite (or the PVD-DLC) have the same volume.

A cell culture substrate of the present disclosure can help to enhance cell culturability by at least 5 times compared to conventional cell culture substrates (for example, glass/polymer Petri dishes), thereby the cell culture substrate can be utilized in disease treatment. The cell culture substrate can be particularly effective in the production of alternative meat products, such as cell-cultured meat.

In particular, the cell culture substrate can help prevent carbon culture layer damage, such as cracks, delamination, and pinholes, during sterilization and cell separation processes even though the carbon culture layer is made of carbon-based materials.

Accordingly, the cell culture substrate is effective in significantly enhancing cell culturability while preventing carbon particles leaked from the damaged carbon culture layer from entering and contaminating cultured cells during the sterilization and cell separation process.

### BRIEF DESCRIPTION OF THE DRAWINGS

FIG. 1 is a cross-sectional view of a high-speed cell culture substrate suitable for sterilization and cell separation processes according to an embodiment of the present disclosure;

FIG. 2 is a cross-sectional view of a high-speed cell culture substrate suitable for sterilization and cell separation processes according to extended embodiments of the present disclosure;

FIG. 3 is a field emission scanning electron microscope (FESEM) analysis photograph of the first carbon allotrope portion according to an embodiment of the present disclosure;

FIG. 4 is an experimental data graphically representing the XPS peak intensities of carbon single bonds (C—C) and carbon double bonds (C═C) in the first carbon allotrope portion corresponding to the plasma density when the first carbon allotrope portion is formed using physical vapor deposition (PVD) according to the present disclosure;

FIG. 5 is an experimental data graphically representing plasma density corresponding to process pressure during a physical vapor deposition (PVD) process;

FIG. 6 is a photograph of the carbon culture layer of Example 1 after sterilization and cell separation processes; and

FIG. 7 is a photograph of the carbon culture layer of Comparative Example 2 after sterilization and cell separation processes.

### DESCRIPTION OF THE PREFERRED EMBODIMENTS

The terms used in this specification are merely used to describe specific embodiments and are not intended to limit the disclosure. Singular expressions include plural expressions unless the context clearly dictates otherwise. In this specification, terms such as ‘comprise’ or ‘have’ are intended to designate the presence of features, numbers, steps, operations, components, parts, or combinations thereof described in the specification, but are not intended to indicate the presence of one or more other features. It should be understood that the terms do not exclude in advance the possibility of the existence or addition of elements, numbers, steps, operations, components, parts, or combinations thereof.

In addition, in this specification, ‘on or above’ means located above or below the target portion, but this does not necessarily mean located above the direction of gravity. In other words, ‘on or above’ as used herein includes not only the case of being located above or below the target part but also the case of being located in front or behind the target part.

Additionally, when a part of a region and plate is said to be ‘on or above’ another part, this does not only mean that the part is in contact with or at a distance ‘directly on or above’ another part, but also that there is yet another part in between the part and another part.

In addition, in this specification, when a component is referred to as ‘connected’ or ‘coupled’ to another component, the component may be directly connected or directly coupled to the other component, but unless there is a contrary description, it should be understood that a component and another component may be connected or coupled by having another component therebetween.

Additionally, in this specification, terms such as first and second may be used to describe various components, but the components should not be limited by the terms. The terms are used only for the purpose of distinguishing one component from another.

Hereinafter, various embodiments, advantages, and features of the present disclosure will be described in detail with reference to the attached drawings.

A cell culture substrate according to the present disclosure is an object used to culture cells by contacting or attaching the cells to a culture layer 20.

The term ‘cell culture’, used herein, may include cell growth or proliferation by contacting or attaching the cells to the culture layer 20 but is not necessarily limited thereto.

The term ‘cell culturability’ used in the present disclosure may include properties including proliferation and growth rate of cells. Therefore, better cell culturability may mean a higher proliferation or growth rate of cells.

In addition, in the case of cells being cultured by attaching to the culture layer 20, cell culturability may further include the cell attachment property to a cell culture surface (for example, cell contact surface to be described later). Therefore, better cell culturability may mean better cell adhesion to the culture layer (that is, cell contact surface).

Cells to be cultured may be stem cells, muscle stem cells, fibroblasts, or nerve cells, but are not necessarily limited thereto.

The present disclosure proposes a cell culture substrate capable of enhancing excellent cell culturability compared to conventional substrates and also preventing carbon-based culture layer 20 damage, such as cracks, delamination, and pinholes, during sterilization and cell separation processes.

Prior to explaining the cell culture substrate, sterilization and cell separation processes in the cell culture process will first be described.

The sterilization process may be necessary to provide a suitable environment for cell growth. The sterilization process removes microorganisms such as bacteria and mold, prevents cell contamination, and promotes cell growth and differentiation.

The sterilization process includes a high temperature/high pressure sterilization, chemical sterilization, and ultraviolet (UV) ray sterilization, and the appropriate sterilization method may be selected depending on the cell type and culture purpose.

The high-temperature sterilization involves removing all microorganisms in cell culture containers using high temperature/high pressure steam at a temperature of 121° C. or higher. After the high-temperature sterilization, the sterilized container goes through a cooling process.

Chemical sterilization involves removing microorganisms from cell culture substrates using alcohol or hydrogen peroxide, and ultraviolet (UV) ray sterilization is a method of removing microorganisms by irradiating the surface of the containers with ultraviolet rays.

The cell separation process involves separating cultured cells from the culture substrates and is generally performed using trypsin.

Trypsin functions to separate cells by decomposing specific proteins on the cell membrane and is mainly used when culturing animal cells.

However, when the culture layer 20 of the cell culture substrate is formed as a carbon-based material, the high temperature/high pressure environment or chemicals used in the sterilization process may cause ‘culture layer damage’ such as cracks, delamination, and pinholes in the carbon culture layer 20.

Additionally, when the carbon culture layer 20 made of carbon is exposed to trypsin during the cell separation process, the carbon culture layer 20 may be damaged, cracked, or peeled off.

That is, during the cell separation process, trypsin decomposes proteins on the surface of the carbon culture layer 20, and as a result, damage to the structure of the carbon membrane, cracks, and detachment may occur.

Likewise, when the culture layer 20 damage occurs during the sterilization and cell separation processes, carbon particles leaked from the culture layer 20 may enter and contaminate the culture cells.

Therefore, when the culture layer 20 of the cell culture substrate is formed as a carbon-based material, it is important to enhance cell culturability, but it is also important to prevent the culture layer 20 damage during the sterilization and cell separation processes.

For this, the present inventors have discovered that when a carbon culture layer 20 is formed by physical vapor deposition (PVD), but on the condition that the carbon culture layer 20 is made of a carbon-based material with specific ranges of hardness, carbon bonding structure (XPS peak intensity ratio), and porosity properties, significant enhancement of cell culturability may be achieved but at the same time culture layer 20 damage, such as cracks, delamination, and pinholes, may be prevented during sterilization and cell separation processes.

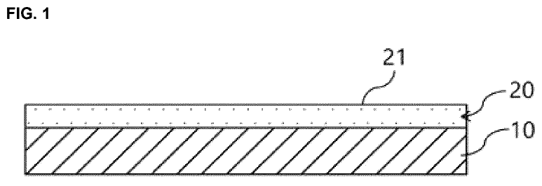

FIG. 1 is a cross-sectional view of a high-speed cell culture substrate suitable for sterilization and cell separation processes according to an embodiment of the present disclosure. Referring to FIG. 1, the high-speed cell culture substrate suitable for the sterilization and cell separation processes includes a base 10 and a culture layer 20.

The base 10 is a structure onto which the culture layer 20 is coated or deposited on one surface. The base may be provided in various forms such as a film, container, substrate, slide, dish, flask, sheet, or block.

The base 10 may be formed of one or more materials selected from synthetic resins, natural resins, glass, silicon, and metals.

The culture layer 20 is formed on the base 10 and has a cell contact surface. The cell contact surface of the culture layer 20 is a surface onto which to-be-cultured cells technically come into contact or attach.

According to one embodiment of the present disclosure, the cell contact surface of the culture layer 20 may be a surface including the top surface of the culture layer 20.

In the culture layer 20, the area including at least the cell contact surface is formed to have a ‘first carbon allotrope portion 21’ which is a portion made of a carbon allotrope.

According to another embodiment, the area including the cell contact surface in the culture layer 20 may be the upper surface of the culture layer 20 and may be continuous with the upper surface, namely, the upper region of the culture layer 20.

When the culture layer 20 is formed so that the first carbon allotrope portion 21 has the following properties in hardness, carbon bonding structure, and pores, the culture layer 20 may significantly enhance cell culturability compared to conventional culture layers while preventing the culture layer 20 damage, such as cracks, peeling, and pinholes during the sterilization and cell separation processes.

### (1) Deposition Method of First Carbon Allotrope Portion

A first carbon allotrope portion 21 is formed by physical vapor deposition (PVD) and may be made of amorphous carbon.

Herein, the term ‘amorphous’ means that the arrangement of a plurality of atoms or molecules lacks periodic regularity, does not make a specific shape, or is formless. Meanwhile, a material that maintains the rules of atomic arrangement over a long distance falls into the category of a crystal.

Therefore, the term ‘amorphous carbon’, used herein, refers to carbon in an amorphous form without a crystal structure.

According to one embodiment of the present disclosure, the first carbon allotrope portion 21 may be formed by physical vapor deposition (PVD) using a plasma source.

The physical vapor deposition (PVD) using a plasma source includes ion plating and sputtering.

The first carbon allotrope portion 21 may be deposited on the upper surface of a base 10 and may be in the form of a thin film, membrane, or film.

According to another embodiment, the first carbon allotrope portion 21 may have a thickness in a range of 5 to 1000 nm, but the thickness is not necessarily limited thereto. For example, the thickness of the first carbon allotrope portion 21 may be in a range of 10 to 1000 nm; 50 to 1000 nm; 100 to 1000 nm; 300 to 1000 nm; 500 to 1000 nm; 5 to 900 nm; 5 to 700 nm; 5 to 500 nm; or 5 to 300 nm.

### (2) Hardness and Resistivity of First Carbon Allotrope Portion

A first carbon allotrope portion 21 is an electrically conductive material having a hardness within the following specific range.

The first carbon allotrope portion 21 is formed to have a greater hardness than graphite, which is another carbon allotrope.

The first carbon allotrope portion 21 is formed to have a weaker hardness than ‘PVD-DLC’ which is diamond-like carbon (DLC) and another carbon allotrope.

The first carbon allotrope portion 21 is formed to have a higher electrical conductivity than the PVD-DLC.

In other words, the first carbon allotrope portion 21 is formed to have a lower resistivity than the PVD-DLC.

The first carbon allotrope portion 21 may have a lower electrical conductivity than graphite.

In other words, the first carbon allotrope portion 21 may have a higher resistivity than graphite.

Herein, graphite is the comparison standard of the first carbon allotrope portion 21 in physical properties, and the maximum hardness and maximum resistivity values of graphite may be reference points for comparison.

Therefore, the first carbon allotrope portion 21 may be a material with a greater hardness than the maximum hardness of graphite and a higher resistivity than the maximum resistivity of graphite.

Herein, PVD-DLC is another comparison standard of the first carbon allotrope portion 21 in physical properties and is formed by physical vapor deposition (PVD). The PVD-DLC is made of amorphous carbon and has properties similar to diamond (that is, carbon bonding structure and physical properties).

The physical vapor deposition (PVD) to form PVD-DLC may be sputtering or ion plating.

When forming PVD-DLC by sputtering, the process gas for use may be an inert gas such as argon (Ar).

PVD-DLC is the comparison standard of the first carbon allotrope portion 21 in physical properties, and the minimum hardness and minimum resistivity values of the PVD-DLC may be reference points for comparison.

Therefore, the first carbon allotrope portion 21 may be a material with a weaker hardness than the minimum hardness of PVD-DLC and a lower resistivity than the minimum resistivity of PVD-DLC.

Specifically, when diamond-like carbon (DLC) is formed by physical vapor deposition (PVD), the diamond-like carbon (DLC) is amorphous and exhibits a hardness of at least 35 GPa, and generally has a strong hardness in a range of 40 to 80 GPa.

PVD-DLC has a resistivity in a range of 1×10¹⁴ to 1×10¹⁶ Ωcm.

A representative example of such PVD-DLC is a tetrahedral amorphous diamond like carbon (Ta-DLC). Such Ta-DLC has a very strong hardness in a range of 60 to 80 GPa. For reference, diamond has a hardness in a range of 70 to 150 GPa.

According to yet another embodiment, PVD-DLC, which is the comparison standard of the first carbon allotrope portion 21 in the hardness and resistivity properties, may be PVD-DLC not doped with other elements or other materials, for example, tetrahedral amorphous diamond like carbon (Ta-DLC).

Graphite, which is another comparison standard of the first carbon allotrope portion 21 in the hardness and resistivity properties, may be pure graphite not doped with other elements or materials.

Graphite, one of the carbon allotropes, is known to have a hardness of 1.5 GPa or less.

Graphite is a conductor with very high electrical conductivity and has a specific resistance in a range of 1×10⁴ to 1×10³ Ωcm.

Therefore, the first carbon allotrope portion 21 is formed to have a hardness greater than 1.5 GPa or less (the hardness range of graphite), and weaker than the hardness range of 35 to 80 GPa (the hardness range of PVD-DLC).

In other words, the first carbon allotrope portion 21 has a hardness greater than 1.5 GPa (the maximum hardness value of graphite), and has a hardness lower than 35 GPa (the minimum hardness value of the PVD-DLC).

Meanwhile, when preventing the carbon culture layer 20 damage, and especially maximizing cell culturability are pursued, conditions related to the XPS peak intensity ratio (I) and pore volume, which will be described later, may be considered together. In this case, the first carbon allotrope portion 21 may be formed to have the following hardness.

According to yet another embodiment, the first carbon allotrope portion 21 may have a hardness in a range of 10 to 29 Gpa, preferably 11 to 28 Gpa; or 12 to 27 Gpa, but the thickness may not be limited thereto. For example, the first carbon allotrope portion 21 may have a hardness in a range of 10 to 28 Gpa; 10 to 27 Gpa; 10 to 26 Gpa; 10 to 25 Gpa; 10 to 24 Gpa; 10 to 29 Gpa; 11 to 29 Gpa; 12 to 29 Gpa; 13 to 29 Gpa; or 14 to 29 Gpa.

Herein, the hardness of the first carbon allotrope portion 21 may be measured with a nano-indenter, which is one of the hardness measurement devices. The nano-indenter may be a nanoindentation tester that executes measurement in compliance with standard test methods ASTM E 2546 to ISO 14577.

In addition, the first carbon allotrope portion 21 may have a resistivity higher than graphite, whose resistivity is in a range of 1×10⁻⁴ to 1×10⁻³ Ωcm, and lower than PVD-DLC, whose resistivity is in a range of 1×10¹⁴ to 1×10¹⁶ Ωcm.

In other words, the first carbon allotrope portion 21 is a conductive material with a resistivity higher than the maximum resistivity of graphite, which is 1×10⁻³ Ωcm, and lower than the minimum resistivity of PVD-DLC, which is 1×10¹⁵ Ωcm.

According to yet another embodiment, the first carbon allotrope portion 21 may have a resistivity in a range of 100 to 2000 Ωcm, but the resistivity is not limited thereto. For example, the resistivity of the first carbon allotrope portion 21 may be in a range of 100 to 1900 Ωcm; 100 to 1800 Ωcm; 100 to 1700 Ωcm; 100 to 1600 Ωcm; 150 to 2000 Ωcm; 200 to 2000 Ωcm; 250 to 2000 Ωcm; or 300 to 2000 Ωcm.

In other words, the first carbon allotrope portion 21 is made of an electrically conductive material with a lower resistivity than that of PVD-DLC, but may have at least 10 times; at least 102 times; or at least 103 times the maximum resistivity of graphite.

Meanwhile, the first carbon allotrope portion 21 may be formed to have the described hardness and resistivity properties by controlling the process conditions for deposition.

For example, when forming the first carbon allotrope portion 21 using sputtering, the first carbon allotrope portion 21 having the described hardness and resistivity properties may be formed by controlling at least a plurality of process conditions selected from the group consisting of current density (W/cm²), plasma density (cm⁻³), process pressure (mTorr), deposition rate (nm/min), electron temperature (eV), and ion flux (mA/cm²).

### (3) Carbon Bonding Structure of First Carbon Allotrope Portion (XPS Peak Intensity Ratio (I)

A first carbon allotrope portion 21 is a material that has both carbon single bonds (C—C) and carbon double bonds (C═C).

According to one embodiment of the present disclosure, the first carbon allotrope portion 21 has a carbon single bond ratio (%) higher than graphite and also has a carbon single bond ratio (%) lower than the PVD-DLC.

According to another embodiment, the first carbon allotrope portion 21 has a carbon double bond ratio (%) lower than the carbon double bond ratio (%) of graphite and also has a carbon double bond ratio (%) higher than the PVD-DLC.

Herein, graphite, which is a comparison standard of the first carbon allotrope portion 21 in the carbon bonding structure (XPS peak intensity ratio (I) property, may be pure graphite not doped with other elements or materials.

PVD-DLC, which is another comparison standard of the first carbon allotrope portion 21 in the carbon bonding structure (XPS peak intensity ratio (I)) property, may be PVD-DLC not doped with other elements or other materials, for example, tetrahedral amorphous diamond like carbon (Ta-DLC).

Meanwhile, the value comparisons between the carbon single/double bond ratio of the first carbon allotrope portion 21 and the carbon single/double bond ratio of graphite and between the carbon single/double bond ratio of the first carbon allotrope portion 21 and the carbon single/double bond ratio of PVD-DLC may be determined through X-ray photoelectron spectroscopy (XPS) analysis.

More specifically, the first carbon allotrope portion 21 may be formed while ensuring that the X-ray photoelectron spectroscopy (XPS) peak intensity ratio (I) calculated according to Equation 1 below, the XPS intensity ratio (I) satisfying Conditional Expression 1 below.

$$XPS peak intensity ratio ( I ) = I 1 / I 2 \qquad [ Equation 1 ]$$

(In Equation 1, I1: XPS peak intensity of the carbon single bond (C—C) of the first carbon allotrope portion, and I2: XPS peak intensity of the carbon double bond (C═C) of the first carbon allotrope portion)

$$0.6 < XPS peak intensity ratio ( I ) < 1.5 \qquad [ Conditional Expression 1 ]$$

Preferably, the first carbon allotrope portion 21 may be formed to have carbon single bonds (C—C) and carbon double bonds (C═C) and to have the XPS peak intensity ratio (I) calculated according to Equation 1 above, the XPS intensity ratio (I) satisfying Conditional Expression 2 below or Conditional Expression 3 below.

$$0.7 < XPS peak intensity ratio ( I ) < 1.4 \qquad [ Conditional Expression 2 ]$$

$$0.8 < XPS peak intensity ratio ( I ) < 1.3 \qquad [ Conditional Expression 3 ]$$

Meanwhile, the first carbon allotrope portion 21 may be formed to have the described XPS peak intensity ratio (I) property by controlling the process conditions for deposition.

For example, when forming the first carbon allotrope portion 21 using sputtering, the first carbon allotrope portion 21 having the described XPS peak intensity ratio (I) property may be formed by controlling the process conditions including plasma density (cm⁻³) and process pressure (mTorr).

### (4) Pore Volume of First Carbon Allotrope Portion

A first carbon allotrope portion 21 has a plurality of pores, and these pores have the following properties.

According to one embodiment, the first carbon allotrope portion 21 may have a pore ratio higher than that of graphite.

Herein, the pore ratio of the first carbon allotrope portion 21 means the total volume occupied by pores in the first carbon allotrope portion 21 (that is, total pore volume) divided by the total volume of the first carbon allotrope portion 21.

The pore ratio of graphite means the total volume occupied by the pores in graphite (that is, total pore volume) divided by the total volume of graphite.

Therefore, when graphite with the same volume as the first carbon allotrope portion is a comparison standard of the first carbon allotrope portion 21, the carbon allotrope portion 21 may be formed to have a greater total pore volume than graphite.

When the first carbon allotrope portion 21 is formed to have the described pore volume property, in relation to this, the first carbon allotrope portion 21 may be formed to have a density lower than graphite. For reference, the theoretical density of graphite is 2.267 g/cm³.

Therefore, according to another embodiment, the first carbon allotrope portion 21 may be formed to have a density in a range of less than 2.267 g/cm³.

According to yet another embodiment, the first carbon allotrope portion 21 may have a density in a range of a maximum of 2.2 g/cm³ and a minimum of 1.05 g/cm³, but the density is not limited thereto. For example, the density of the first carbon allotrope portion 21 may be in a range of 2.15 g/cm³ or less and 1.05 g/cm³ or more; 2.1 g/cm³ or less and 1.05 g/cm³ or more; 2.05 g/cm³ or less and 1.05 g/cm³ or more; 2.0 g/cm³ or less and 1.05 g/cm³ or more; 2.2 g/cm³ or less and 1.1 g/cm³ or more; 2.2 g/cm³ or less and 1.15 g/cm³ or more; 2.2 g/cm³ or less and 1.2 g/cm³ or more; or 2.2 g/cm³ or less and 1.25 g/cm³ or more.

According to yet another embodiment, the first carbon allotrope portion 21 may have a pore ratio higher than PVD-DLC.

Herein, the pore ratio of PVD-DLC means the total volume occupied by the pores in PVD-DLC (that is, total pore volume) divided by the total volume of PVD-DLC.

Therefore, when PVD-DLC with the same volume as the first carbon allotrope portion is a comparison standard of the first carbon allotrope portion 21, the carbon allotrope portion may be formed to have a greater total pore volume than that of PVD-DLC.

In addition, when the first carbon allotrope portion 21 is formed to have the described pore volume property, in relation to this, the first carbon allotrope portion 21 may be formed to have a density lower than PVD-DLC.

Herein, PVD-DLC, which is a comparison standard of the first carbon allotrope portion 21 in the pore volume property, may be PVD-DLC not doped with other elements or other materials, for example, tetrahedral amorphous diamond like carbon (Ta-DLC).

Ta-DLC may have a density similar to diamond and specifically has a density in a range of about 2.39 to 3.26 g/cm³. For reference, the theoretical density of diamond is 3.515 g/cm³.

Graphite, which is another comparison standard of the first carbon allotrope portion 21 in the pore volume property, may be pure graphite not doped with other elements or materials.

In other words, the first carbon allotrope portion 21 according to yet another embodiment of the present disclosure may be formed to have a lower density than PVD-DLC, and may further have a lower density than graphite, which has a lower density than PVD-DLC.

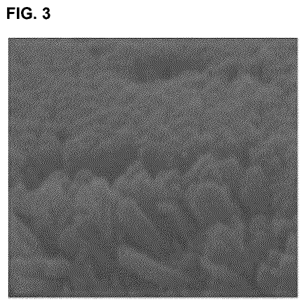

FIG. 3 is a field emission scanning electron microscope (FESEM) analysis photograph of the first carbon allotrope portion according to an embodiment of the present disclosure. As seen in FIG. 3, the first carbon allotrope portion is formed with a plurality of pores in a huge size, and the first carbon allotrope portion has a pore ratio higher than graphite as well as PVD-DLC.

In addition, when the first carbon allotrope portion 21 is formed to have the described pore volume property, the first carbon allotrope portion 21 may be formed to have a density lower than graphite as well as PVD-DLC.

Meanwhile, the first carbon allotrope portion 21 may be formed to have the described pore volume property by controlling the process conditions for deposition.

For example, when forming the first carbon allotrope portion 21 using sputtering, the first carbon allotrope portion 21 having the described pore volume property may be formed by controlling at least a plurality of process conditions selected from the group consisting of current density (W/cm²), plasma density (cm⁻³), process pressure (mTorr), deposition rate (nm/min), electron temperature (eV), and ion flux (mA/cm²).

### (5) Process Conditions (Plasma Density and Working Pressure)

According to one embodiment of the present disclosure, a first carbon allotrope portion 21 may be formed by sputtering deposition. In this case, the first carbon allotrope portion 21 may be formed by controlling plasma density and working pressure to meet specific process conditions.

In other words, when forming the first carbon allotrope portion 21 by sputtering under a process atmosphere with a plasma density and process pressure within the following ranges, carbon-based materials having the described hardness, carbon bonding structure (XPS peak intensity ratio), and pore volume properties may be formed.

According to another embodiment, when forming the first carbon allotrope portion 21 by sputtering, the working pressure may be greater than 1 mTorr and less than 130 mTorr and preferably may be in a range of 3 to 100 mTorr; 3 to 70 mTorr; 3 to 50 mTorr; or 3 to 20 mTorr.

According to yet another embodiment, when forming the first carbon allotrope portion 21 by sputtering, plasma density may be greater than 1.0×10⁹ cm⁻³ and less than 1.0×10¹¹ cm⁻³ and preferably may be in a range of 2.0×10⁹ to 9.0×10¹⁰ cm⁻³.

Herein, plasma density may refer to the density of ionized particles in a sputtering process.

According to yet another embodiment, the first carbon allotrope portion 21 may be formed by magnetron sputtering. In this case, magnetron sputtering may be unbalanced magnetron sputtering (UBMS).

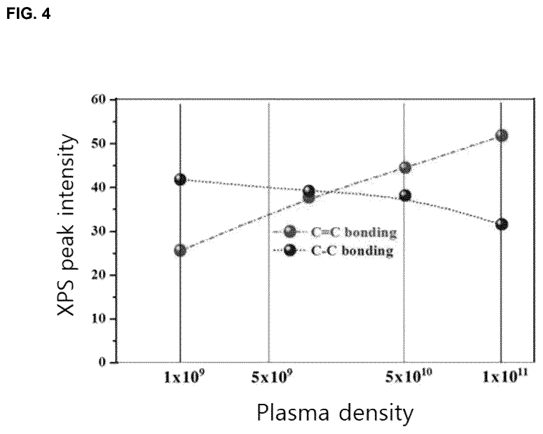

FIG. 4 is an experimental data graphically representing the XPS peak intensities of carbon single bonds (C—C) and carbon double bonds (C═C) in the first carbon allotrope portion 21 corresponding to the plasma density when the first carbon allotrope portion 21 is formed using physical vapor deposition (PVD) according to the present disclosure.

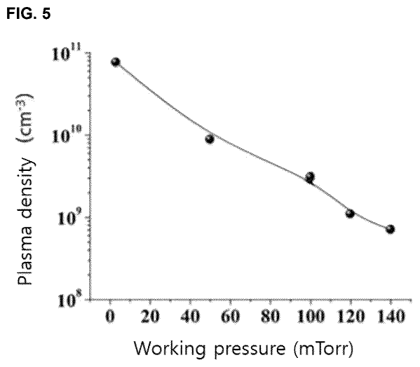

FIG. 5 is an experimental data graphically representing plasma density corresponding to process pressure during a physical vapor deposition (PVD) process.

Referring to FIGS. 4 and 5, when forming the first carbon allotrope portion 21 by physical vapor deposition (PVD) using a plasma source while controlling the plasma density of the first carbon allotrope portion 21 to be greater than 1.0×10⁹ cm⁻³ and less than 1.0×10¹¹ cm⁻³, it may be conformed that the first carbon allotrope portion 21 having the described ‘carbon bonding structure (XPS peak intensity ratio (I)),’ which is a condition satisfying Conditional Expression 1, is formed.

According to yet another embodiment, physical vapor deposition (PVD) using a plasma source may be sputtering.

In addition to this plasma density, by controlling current density (W/cm²), working pressure (mTorr), deposition rate (nm/min), electron temperature (eV), and ion flux (mA/cm²) under specific conditions, the first carbon allotrope portion 21 having hardness, resistivity, and pore volume properties within the described specific range may be formed.

### (6) Extended Embodiments

According to one extended embodiment of the present disclosure, the first carbon allotrope portion 21 may be carbon materials doped with nitrogen (N).

When doping the described first carbon allotrope portion 21 with nitrogen (N), during the sputtering process to form a culture layer 20, nitrogen gas may be injected below a certain threshold relative to the overall working pressure, thereby a nitrogen-doped first carbon allotrope portion 21 may be formed.

According to another embodiment, during the sputtering process to form the culture layer 20, the partial pressure of nitrogen gas may be in a range of up to 10% (that is, 10% or less) relative to the total working pressure and may be preferably in a range of 9% or less; 8% or less; 7% or less; 6% or less; or 5% or less.

This is because when the partial pressure of nitrogen gas is beyond 10%, the stress increases, making it difficult to form a carbon film (that is, the culture layer 20). In addition, the film properties deteriorate, and the adhesion and attachment (that is, bonding strength) of the culture layer 20 to the base 10 significantly decrease.

For reference, when there is an area where the adhesion is not uniform or the bonding strength is weak between the base 10 and the culture layer 20, the culture layer 20 may be peeled off from the base 10, or cracks may occur in that area. These issues may be factors that reduce cell culturability.

According to yet another embodiment, the nitrogen may be contained in the first carbon allotrope portion 21 in an amount of up to 10% by weight (that is, 10% by weight or less), and preferably in an amount of 9% by weight or less; 8% by weight or less; 7% by weight or less; 6% by weight or less; 5% by weight or less; or 4% by weight or less.

This is because when nitrogen is contained in an amount of more than 10% by weight, it becomes difficult to form a carbon film (that is, culture layer 20), and the adhesion and attachment (that is, bonding strength) of the culture layer 20 to the base 10 significantly decrease, and ultimately these issues reduce cell culturability.

Therefore, to enhance the cell culturability of the cell culture substrate, the culture layer 20 is required to be able to be formed on the base 10 with uniform adhesion and strong bonding strength throughout. For this, the first carbon allotrope portion 21 may be formed with the use of nitrogen gas within the described range of partial pressure or nitrogen content.

Likewise, when doping the first carbon allotrope portion 21 with nitrogen within a specific range of nitrogen partial pressure or nitrogen content, it is possible to prevent the culture layer 20 damage during sterilization and cell separation processes, while maximizing cell culture performance.

In addition, through such nitrogen doping and doping amount control, users may control the first carbon allotrope portion 21 to have hardness, carbon bonding structure, and pore properties optimally suitable for the cell type or subject to be cultured.

According to yet another extended embodiment, the first carbon allotrope portion 21 may be a carbon material doped with hydrogen (H).

In other words, depending on the cell type or subject the user wishes to culture, the first carbon allotrope portion 21 may be doped with hydrogen (H). At this time, like nitrogen doping, a hydrogen doping amount may be within a range considering the adhesion and bonding strength of the culture layer 20 to the base 10.

According to yet another embodiment, the first carbon allotrope portion may be doped with hydrogen, and the hydrogen-doped first carbon allotrope portion may be formed by injecting hydrogen gas during a physical vapor deposition (PVD) process. At this time, in the physical vapor deposition (PVD) process, the partial pressure of hydrogen gas may be in a range of 10% or less relative to the total working pressure, and preferably may be in a range of 9% or less; 8% or less; 7% or less; 6% or less; or 5% or less.

According to yet another embodiment, the hydrogen gas may be contained in the first carbon allotrope portion 21 in an amount of up to 10% by weight (that is, 10% by weight or less), and preferably in an amount of 9% by weight or less; 8% by weight or less; 7% by weight or less; 6% by weight or less; 5% by weight or less; or 4% by weight or less.

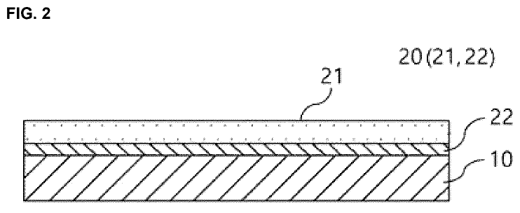

FIG. 2 is a cross-sectional view of a high-speed cell culture substrate suitable for sterilization and cell separation processes according to extended embodiments of the present disclosure.

Referring to FIG. 2, the culture layer 20 according to an expanded embodiment of the present disclosure may further include a second carbon allotrope portion 22′ which is another portion made of a carbon allotrope.

The second carbon allotrope portion 22 is formed between the first carbon allotrope portion 21 and the base 10 in the culture layer 20.

The second carbon allotrope portion 22 is formed to have a hardness higher than the first carbon allotrope portion 21 and a hardness equal to or lower than the hardness of the described PVD-DLC.

According to yet another embodiment, the second carbon allotrope portion 22 may have a carbon single bond ratio higher than the first carbon allotrope portion 21.

According to yet another embodiment, the second carbon allotrope portion 22 may have a carbon double bond ratio lower than the first carbon allotrope portion 21.

When the culture layer 20 further includes the second carbon allotrope portion 22, the adhesion and bonding strength thereof to the base 10 may further increase, and excellent cell culture properties may be ensured. In particular, maximizing the ability to suppress the culture layer damage in sterilization and cell separation processes is possible.

Meanwhile, the second carbon allotrope portion 22 may be formed to have a hardness gradient region within the described hardness range conditions.

According to yet another embodiment, the second carbon allotrope portion 22 may be formed to include a portion having a hardness gradient in which the hardness gradually increases from the first carbon allotrope portion 21 toward the base 10.

Hereinafter, the cell culture substrate of the present disclosure as described above will be further described based on examples and comparative examples. However, the following examples only illustrate the content of the present disclosure, and the scope of the present disclosure is not limited by the following examples.

### (1) Examples 1 to 3

In Examples 1 to 3, cell culture substrates satisfy all the properties of the first carbon allotrope portion of the present disclosure.

In other words, the culture layers according to Examples 1 to 3 were prepared in a manner such that all the hardness, XPS peak intensity ratio, and pore volume of the first carbon allotrope portion fell within the physical property range presented in the present disclosure.

### (2) Comparative Examples 2 to 5

In Comparative Examples 2 to 5, cell culture substrates do not satisfy at least one of the properties of the first carbon allotrope portion of the present disclosure.

In other words, the culture layers according to Comparative Examples 2 to 5 were prepared in a manner such that at least two properties of the first carbon allotrope portion, including hardness, carbon bonding structure (XPS peak intensity ratio), and pore volume, fell outside the physical property range presented in the present disclosure.

### (3) Physical Property Measurement Methods

### (3-1) Hardness

The hardness of each culture layer was measured using a nano-indenter that complies with ISO 14577.

### (3-2) XPS Peak Intensity Ratio (I)

An XPS analysis was performed for each culture layer using a MultiLab 2000 spectrometer (Thermo Electron Corporation, UK).

Using the XPS data analyzed this way, the XPS peak intensity ratios (I) for Examples 1 to 3 and Comparative Examples 2 to 5 were calculated according to the mentioned [Equation 1].

### (3-3) Pore Volume

After measuring each culture layer's density, the corresponding pore volume properties were calculated based on the density.

### (4) Test Items

### (4-1) Cell Culturability

L-929 cells were attached to the upper surface (that is, cell contact surface) of the culture layer of the cell culture substrate according to Examples 1 to 3 and Comparative Examples 1 to 5. The culture rates (%) at 1 day, 3 days, and 5 days were measured and averaged, and then the average value was compared with Comparative Example 1.

Among the items in Tables 1 and 2, the written ‘culture rate (fold)’ is calculated as the multiple of the average culture rate (%) of each Example relative to the average culture rate (%) of Comparative Example 1.

In other words, the average value of the culture rate of Comparative Example 1 was written being converted to ‘1’, and then after the ‘average value of culture rate’ of each test example was converted at the same ratio accordingly, the multiple of the ‘average culture rate’ of each Example relative to the average culture rate of Comparative Example 1 was calculated and described in the ‘culture rate (fold)’ item in Tables 1 and 2.

In Comparative Example 1, a cell culture substrate commonly used in cell culture was used. Specifically, the cell culture substrate did not have a carbon culture layer like cell culture substrates shown in Examples 1 to 3 and Comparative Examples 2 to 5. Still, a conventional Petri dish made of glass was purchased and used as the cell culture substrate in Comparative Example 1.

Cell activity, such as culture speed and absorbance of L-929 cells attached to the cell culture substrate, was determined by using an XTT cell proliferation assay kit. In this cell culturability test, untreated L-929 cells derived from rat fibroblasts were cultured.

To confirm the culturability of the cells cultured on the cell culture substrate, the culturability was measured by percentage using a fluorescence spectrophotometer at a wavelength of 450 nm.

### (4-2) Confirmation of Culture Layer Damage

After performing sterilization and cell separation processes on each cell culture substrate according to Examples 1 to 3 and Comparative Examples 1 to 5, damage to the culture layer of the cell culture substrates (that is, peeling and cracks) was checked and listed in Table 1.

### (5) Test Results

In Examples 1 to 3 and Comparative Examples 1 to 5, cell culturability (that is, cell culture rate (%)) and culture layer damage in the sterilization and cell separation processes were tested and are shown in Tables 1 and 2 below.

TABLE 1

| Division | Example 1 | Example 2 | Example 3 |
|---|---|---|---|
| Hardness (GPa) | 10.4 | 19.2 | 28.7 |
| XPS peak ratio (I) | 0.68 | 0.97 | 1.42 |
| Total pore volume | Larger than graphite | Larger than graphite | Larger than graphite |
| | and PVD-DLC | and PVD-DLC | and PVD-DLC |
| Culture rate_5-day | 4.5 fold | 4.1 fold | 3.9 fold |
| average (fold) | | | |
| Culture layer | Not damaged | Not damaged | Not damaged |
| damage | | | |

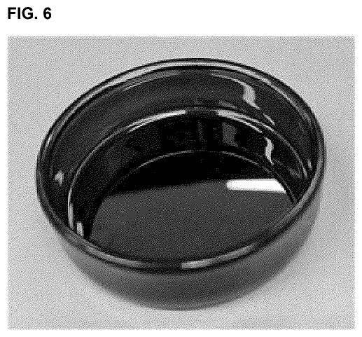

As shown in Examples 1 to 3 of Table 1, when the hardness, XPS peak intensity ratio, and pore volume of the culture layer (that is, a first carbon allotrope portion) were within the physical property range of the present disclosure, compared to conventional Petri dish (Comparative Example 1), cell culturability was significantly enhanced, and even when sterilization and cell separation processes were performed, culture layer damage, such as peeling and cracks, did not occur as shown in FIG. 6.

For reference, FIG. 6 is a photograph of the culture layer of Example 1 after sterilization and cell separation processes.

TABLE 2

| | Comparative | Comparative | Comparative | Comparative | Comparative |
|---|---|---|---|---|---|
| Division | Example 1 | Example 2 | Example 3 | Example 4 | Example 5 |
| Hardness | Conventional | 0.8 | 1.46 | 37.2 | 49.3 |
| (GPa) | Petri dish | | | | |
| XPS peak | | 0.23 | 0.35 | 2.83 | 4.9 |
| ratio (I) | | | | | |
| Total pore | | Larger than | Larger than | Smaller than | Smaller than |
| volume | | PVD-DLC | PVD-DLC | graphite | graphite |
| Culture | 1 | 4.9 fold | 4.8 fold | 1.7 fold | 1.4 fold |
| rate_5-day | | | | | |
| average | | | | | |
| (fold) | | | | | |
| Culture | Not | Damaged | Damaged | Not | Not |
| layer | damaged | | | damaged | damaged |
| damage | | | | | |

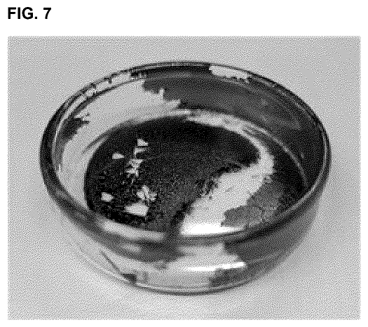

As shown in Examples 1 to 3 in Table 1, in Comparative Examples 2 and 3, cell culturability was significantly enhanced compared to conventional Petri dish (Comparative Example 1), but culture layer damage, including peeling and cracks, occurred as shown in FIG. 7, especially when performing sterilization and cell separation processes.

For reference, FIG. 7 is a photograph of the carbon culture layer of Comparative Example 2 after sterilization and cell separation processes.

Meanwhile, even when the cell culture substrates of Comparative Examples 4 and 5 were subjected to sterilization and cell separation processes, culture layer damage did not occur. However, in tests with the cell culture substrates of Comparative Examples 4 and 5, a slight increase in cell culturability was shown compared to the substrate of Comparative Example 1 (that is, a conventional cell culture substrate without a carbon culture layer).

Although preferred examples of the present disclosure have been described and illustrated above using specific terminology, such terminology is only intended to clearly describe the present disclosure. It is obvious that various changes and changes can be made to the examples and described terms of the present disclosure without departing from the technical spirit and scope of the following claims. These modified examples should not be understood individually outside of the spirit and scope of the present disclosure but should be regarded as falling within the scope of the claims of the present disclosure.

What is claimed is:

1. A high-speed cell culture substrate suitable for sterilization and cell separation processes, the high-speed cell culture substrate comprising:
a base; and
a culture layer formed on the base and having a ‘cell contact surface’ which is a surface to which cells come into contact or attach,
wherein in the culture layer, an area including at least the cell contact surface has a ‘first carbon allotrope portion’ which is a portion made of a carbon allotrope,
the first carbon allotrope portion is formed by physical vapor deposition (PVD),
the first carbon allotrope portion is an electronic conductive material, and
the first carbon allotrope portion has a higher hardness than graphite, which is another carbon allotrope, and a lower hardness than ‘PVD-DLC’ formed by PVD, which is a diamond-like carbon (DLC) and another carbon allotrope.

2. The high-speed cell culture substrate of claim 1, wherein the graphite has a hardness in a range of 1.5 Gpa or less,
the PVD-DLC has a hardness in a range of 35 Gpa or more, and
the first carbon allotrope portion has a greater hardness than the maximum hardness of the graphite and a weaker hardness than the minimum hardness of the PVD-DLC.

3. The high-speed cell culture substrate of claim 1, wherein the first carbon allotrope portion has a hardness in a range of 10 to 29 Gpa.

4. The high-speed cell culture substrate of claim 1, wherein the first carbon allotrope portion has a hardness in a range of 11 to 28 Gpa.

5. The high-speed cell culture substrate of claim 1, wherein the first carbon allotrope portion has a carbon single bond (C—C) and a carbon double bond (C═C),
the first carbon allotrope portion has a carbon single bond ratio higher than the graphite and lower than the PVD-DLC, and
the first carbon allotrope portion has a carbon double bond ratio lower than the graphite and higher than the PVD-DLC.

6. The high-speed cell culture substrate of claim 1, wherein the first carbon allotrope portion has a carbon single bond (C—C) and a carbon double bond (C═C),
the first carbon allotrope portion is a material with an XPS peak intensity ratio (I) calculated according to Equation 1 below, the XPS intensity ratio (I) satisfying Conditional Expression 1 below.
$$XPS peak intensity ratio ( I ) = I 1 / I 2 \qquad [ Equation 1 ]$$
(In Equation 1, I1: XPS peak intensity of the carbon single bond (C—C), and I2: XPS peak intensity of the carbon double bond (C═C))
$$0.6 < XPS peak intensity ratio ( I ) < 1.5 \qquad [ Conditional Expression 1 ]$$

7. The high-speed cell culture substrate of claim 6, wherein the first carbon allotrope portion is a material with an XPS peak intensity ratio (I) calculated according to Equation 1 above, the XPS intensity ratio (I) satisfying Conditional Expression 2 below.
$$0.7 < XPS peak intensity ratio ( I ) < 1.4 \qquad [ Conditional Expression 2 ]$$

8. The high-speed cell culture substrate of claim 1, wherein the first carbon allotrope portion has a plurality of pores, and
the first carbon allotrope portion is a material with a larger total pore volume than the graphite when the first carbon allotrope portion and the graphite have the same volume.

9. The high-speed cell culture substrate of claim 1, wherein the first carbon allotrope portion has a plurality of pores, and
the first carbon allotrope portion is a material with a larger total pore volume than the PVD-DLC when the first carbon allotrope portion and the PVD-DLC have the same volume.

10. The high-speed cell culture substrate of claim 1, wherein the first carbon allotrope portion is doped with nitrogen (N).

11. The high-speed cell culture substrate of claim 10, wherein the nitrogen-doped first carbon allotrope portion is formed by injecting a nitrogen gas during a physical vapor deposition (PVD) process to form the first carbon allotrope portion, and
the partial pressure of the nitrogen gas in the PVD process is 10% or less relative to the overall working pressure.

12. The high-speed cell culture substrate of claim 10, wherein the nitrogen is contained in the first carbon allotrope portion in an amount of 10% by weight or less.

13. The high-speed cell culture substrate of claim 1, wherein the first carbon allotrope portion has a lower electronic conductivity than the graphite, and
the first carbon allotrope portion has a higher electronic conductivity than the PVD-DLC.

14. The high-speed cell culture substrate of claim 1, wherein the first carbon allotrope portion is formed by a sputtering process with a plasma density of more than 1.0×10⁹ cm⁻³ and less than 1.0×10¹¹ cm⁻³.

15. The high-speed cell culture substrate of claim 1, wherein the cell contact surface comprises an upper surface of the culture layer.

16. The high-speed cell culture substrate of claim 1, wherein the culture layer further comprises a ‘second carbon allotrope portion’ which is another portion made of a carbon allotrope,
wherein the second carbon allotrope portion is formed between the first carbon allotrope portion and the base,
the second carbon allotrope portion has a greater hardness than the first carbon allotrope portion, and
the second carbon allotrope portion has a hardness equal to or lower than the PVD-DLC.

17. The high-speed cell culture substrate of claim 1, wherein the second carbon allotrope portion has a higher carbon single bond ratio than the first carbon allotrope portion, and
the second carbon allotrope portion has a lower carbon double bond ratio than the first carbon allotrope portion.

18. The high-speed cell culture substrate of claim 1, wherein the second carbon allotrope portion has a hardness gradient region in which the hardness gradually increases from the first carbon allotrope portion toward the base.
